(19) United States
(12) Patent Application Publication
(10) Pub. No.: US 2016/0001048 A1
(43) Pub. Date: Jan. 7, 2016

(54) GUIDEWIRE

(71) Applicant: ASAHI INTECC CO., LTD., Nagoya-shi (JP)

(72) Inventor: Tadahiro KOIKE, Ama-shi (JP)

(21) Appl. No.: 14/736,586

(22) Filed: Jun. 11, 2015

(30) Foreign Application Priority Data
Jul. 2, 2014 (JP) 2014-136428

(51) Int. Cl. A61M 25/09

(52) U.S. Cl. CPC A61M 25/09033; A61M 2025/09083; A61M 2025/09191; A61M 2025/0915

(57) ABSTRACT

A guidewire is capable of pressing against a lesion without bending excessively, thereby ensuring reliable use. The guidewire includes a shaft, an outer coil provided around a distal portion of the shaft, a first inner coil provided within a distal portion of the outer coil, and a second inner coil provided proximal to the first inner coil within the outer coil. A first joining member joins the shaft to a proximal end of the first inner coil, or a proximal portion of the first inner coil distal to the proximal end of the first inner coil. A second joining member joins the shaft to a distal end of the second inner coil, or a distal portion of the second inner coil proximal to the distal end of the second inner coil. The first joining member and the second joining member are spaced from each other in a longitudinal direction of the guidewire.

### CROSS REFERENCE TO RELATED APPLICATION

This application claims priority to Japanese Application No. 2014-136428 filed on Jul. 2, 2014, the content of which is incorporated by reference herein in its entirety.

### BACKGROUND

The disclosed embodiments relate to a medical device. Specifically, the disclosed embodiments relate to a guidewire to be inserted into body cavities for purposes of treatment or examination.

Various guidewires have been developed to guide a catheter or the like that is inserted into either a tubular organ (such as a blood vessel, a digestive tract, or a ureter) or body tissue for purposes of treatment or examination. For example, Japanese Patent Application Publication No. 08-317989 (JP 8-317989 A) describes a guidewire that includes a core wire, an outer coil provided around an outer circumference of a distal portion of the core wire, and an inner coil provided within the outer coil.

However, when a distal portion of the guidewire of JP 8-317989 A presses against a hard lesion (for example, in a lower limb region), the distal portion bends to an extreme degree. If the bent guidewire is inserted even deeper into the tubular organ or body tissue, the distal portion may bend to an even greater degree. As a result, the bent distal portion will undergo plastic deformation, making it difficult to manually restore the shape of the distal portion. In other words, conventional guidewires have room for improvement in terms of preventing the excessive bending that occurs when a guidewire presses against a hard lesion.

### SUMMARY

An object of the disclosed embodiments is to provide a guidewire in which excessive bending of the guidewire is suppressed even when the guidewire presses against a hard lesion. Reliable use of the guidewire can therefore be ensured.

In order to achieve this and/or other objects, a guidewire of the disclosed embodiments has the following characteristics.

A guidewire of the disclosed embodiments includes a shaft, an outer coil wound around a distal portion of the shaft, and a first inner coil and a second inner coil provided within the outer coil. The first inner coil is provided within a distal portion of the outer coil, and the second inner coil is provided proximal to the first inner coil. A proximal portion of the first inner coil is joined to the shaft via a first joining member, and a distal portion of the second inner coil is joined to the shaft via a second joining member. The first joining member and the second joining member are spaced from each other at a non-zero distance in a longitudinal direction of the guidewire. That is, there is a gap between the first inner coil and the second inner coil in the longitudinal direction.

Usually, when torque is applied to a handling portion of a guidewire and the outer coil is consequently tightened by winding, the outer coil deforms inwardly, and the diameter of the outer coil is reduced. When the outer coil is excessively deformed, an elemental wire constituting the outer coil may become displaced, sliding between adjacent elemental wires or adjacent windings of the elemental wire (in a single-strand coil). However, the inner coils are provided so as to support the outer coil and prevent excessive deformation. In particular, the first inner coil and the second inner coil are spaced from each other. Therefore, the first inner coil and the second inner coil do not interfere with each other when the outer coil deforms inwardly, contacting the inner coils.

As discussed above, the distal portion of the guidewire will bend when it presses against a hard calcified lesion, for example. In the guidewire of the disclosed embodiments, the first joining member and the second joining member come into contact with each other when the distal portion of the guidewire is bent, preventing further movement of the first and second inner coils and thus suppressing excessive bending. As a result, plastic deformation of the distal portion of the guidewire is prevented. This ensures that the guidewire can be reliably and consistently used, even when the guidewire is inserted into a hard calcified lesion.

The guidewire may further include a third inner coil provided within the outer coil and provided proximal to the second inner coil. A proximal portion of the second inner coil is joined to the shaft via a third joining member, a distal portion of the third inner coil is joined to the shaft via a fourth joining member, and a proximal portion of the third inner coil is joined to the shaft via a fifth joining member. The third joining member and the fourth joining member are spaced from each other at a non-zero distance in a longitudinal direction. That is, there is a gap between the third inner coil and the fourth inner coil in the longitudinal direction.

The ends of the inner coils may terminate at the joining members, or might project beyond the joining members. For example, the first joining member may be provided at a proximal end of the first inner coil, and the second joining member may be provided at a distal end of the second inner coil. Alternatively, the proximal end of the first inner coil might project proximally beyond the first joining member, and the distal end of the second inner coil might project distally beyond the second joining member. In other words, the first joining member may be provided at a position distal to the proximal end of the first inner coil, but still within the proximal portion of the first inner coil. Likewise, the second joining member may be provided at a position proximal to the distal end of the second inner coil, but still within the distal portion of the second inner coil.

The outer diameter of the outer coil may be uniform in the longitudinal direction, or may increase from the distal end of the outer coil toward the proximal end of the outer coil. Additionally, an outer diameter of the first inner coil may be substantially the same as an outer diameter of the second inner coil, or the outer diameter of the second inner coil may be greater than the outer diameter of the first inner coil.

The guidewire may include both an outer coil in which the outer diameter increases from the distal end toward the proximal end, and also inner coils in which the outer diameter of the second inner coil is greater than the outer diameter of the first inner coil. In this case, the outer diameters of the first inner coil and the second inner coil may be selected to correspond to the shape of the outer coil. Accordingly, the difference in the outer diameter of the first inner coil and the outer diameter of the second inner coil can suppress inward deformation of the outer coil.

Because of the difference in diameters, the proximal end of the first inner coil and the distal end of the second inner coil part are spaced from each other in a direction perpendicular to the longitudinal direction, and thus do not interfere with each other. Thus, the distal end of the second inner coil can be efficiently joined to the shaft after joining the proximal end of the first inner coil without any interference occurring between the site of joining of the second inner coil (the distal end of the second inner coil) and the proximal end of the first inner coil.

The inner coils may each be a single-strand coil formed by winding an elemental wire in a helical fashion. Alternatively, each of the inner coils may be formed by winding a plurality of elemental wires together, or by winding a plurality of stranded wires together, each of the stranded wires being formed by twisting a plurality of elemental wires together.

When the inner coils are formed by winding a plurality of elemental wires together, the joining material used to join the inner coils to the shaft can preferentially penetrate into gaps between the elemental wires along the longitudinal direction by capillary action. As a result, the resulting joining member (e.g., the first joining member or the second joining member) has an adequate volume and rigidity.

Therefore, when the distal portion of the guidewire presses against a hard calcified lesion and bends, the first joining member and the second joining member come into contact with each other, preventing further movement of the first and second inner coils and thus suppressing excessive bending. As a result, plastic deformation of the distal portion of the guidewire is prevented even more reliably.

The outer coil may be a single-strand coil formed by winding an elemental wire in a helical fashion. Alternatively, the outer coil may be formed by winding a plurality of stranded wires in a helical fashion, each of the stranded wires being formed by twisting a plurality of elemental wires together. In the stranded wires, the elemental wires are capable of slightly moving relative to each other, providing the outer coil with excellent flexibility and an adequate restoring capacity. As a result, when the distal portion of the guidewire presses against a hard calcified lesion and bends, the shape of the guidewire can be manually restored with ease, which makes it possible to use the guidewire consistently.

Furthermore, when torque is applied to a handling portion of the guidewire, not only are the elemental wires pressed against each other, but also the stranded wires are pressed against each other, increasing contact pressure and enhancing close contact. This improves torque transmission and ensures excellent maneuverability of the guidewire.

### BRIEF DESCRIPTION OF THE DRAWINGS

FIG. 1 is an expanded view of a partial cross-section of a guidewire according to the disclosed embodiments.

FIG. 2 is an expanded view of a partial cross-section of the guidewire of FIG. 1 when bent.

FIG. 3 is an expanded view of a partial cross-section of a guidewire according to the disclosed embodiments.

FIG. 4 is an expanded view of a partial cross-section of a guidewire according to the disclosed embodiments.

FIG. 5 is an expanded view of a partial cross-section of a guidewire according to the disclosed embodiments.

FIG. 6 is an expanded view of a partial cross-section of a guidewire according to the disclosed embodiments.

FIG. 7 is an expanded view of a partial cross-section of a guidewire according to the disclosed embodiments.

FIG. 8 is an expanded view of a partial cross-section of a guidewire according to the disclosed embodiments.

FIG. 9 is a perspective view of an inner coil of the guidewire shown in FIG. 8.

FIG. 10 is an expanded view of a partial cross-section of a guidewire according to the disclosed embodiments.

FIG. 11 is a cross-sectional view taken along line A-A of FIG. 10.

FIG. 12 is an expanded view of a partial cross-section of a guidewire according to the disclosed embodiments.

FIG. 13 is a cross-sectional view taken along line B-B of FIG. 12.

### DETAILED DESCRIPTION OF EMBODIMENTS

Guidewires of the disclosed embodiments are explained with reference to the drawings.

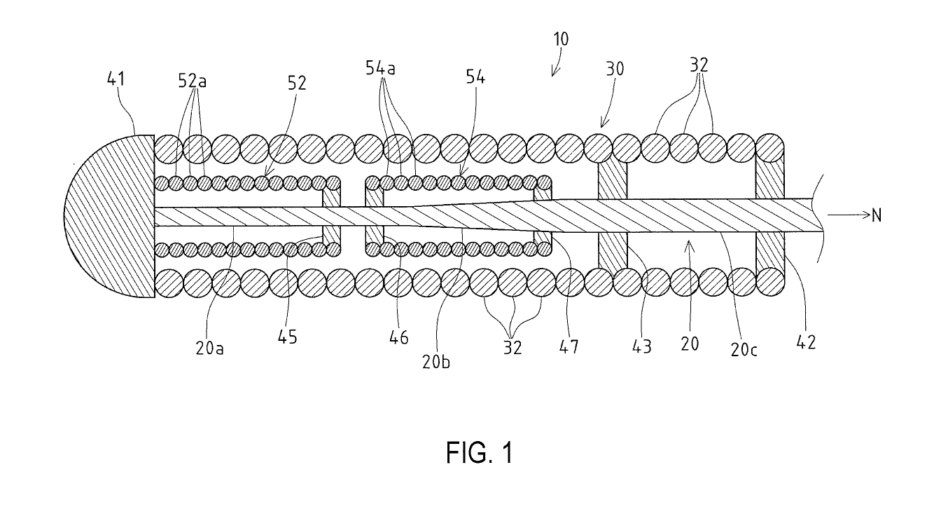

FIG. 1 is an expanded view of a partial cross-section of a guidewire 10 according to the disclosed embodiments. In FIGS. 1 to 8, 10, and 12, a distal end to be inserted into a body is shown on the left side, and a proximal end to be handled by a manipulator such as a doctor is shown on the right side.

A guidewire 10 shown in FIG. 1 is used, for example, in treating blood vessels of a lower limb by the Cross-Over technique. The guidewire 10 includes a shaft 20, an outer coil 30 provided around an outer circumference of a distal portion of the shaft 20, and a first inner coil 52 and a second inner coil 54 provided within the outer coil 30.

The shaft 20 is explained first. The shaft 20 includes a smaller-diameter portion 20a, a tapered portion 20b, and a greater-diameter portion 20c, in this order from a distal end of the shaft toward a proximal end of the shaft. The smaller-diameter portion 20a is the most distal portion of the shaft 20 and the most flexible part of the shaft 20. The smaller-diameter portion 20a may be formed flat by pressing. The tapered portion 20b is tapered with a circular cross section, with its diameter decreasing toward its distal end. The greater-diameter portion 20c has a diameter that is greater than a diameter of the smaller-diameter portion 20a.

The material used to form the shaft 20 is not particularly limited and includes a stainless steel (SUS304), a super-elastic alloy such as Ni—Ti alloys, piano wire, a cobalt-based alloy, or the like.

Next, the outer coil 30 is explained. The outer coil 30 of the guidewire 10 is a single-strand coil formed by winding an elemental wire 32 in a helical fashion. The outer diameter of the outer coil 30 is substantially uniform in the longitudinal direction N.

The material used to form the outer coil 30 is not particularly limited and includes stainless steels such as martensitic stainless steel, ferritic stainless steel, austenitic stainless steel, austenitic-ferritic duplex stainless steel, and precipitation hardened stainless steel; super-elastic alloys such as Ni—Ti alloys; and metals radiopaque to X-rays, such as platinum, gold, tungsten, tantalum, iridium, and alloys thereof.

As shown in FIG. 1, a distal end of the outer coil 30 is fixed to a distal end of the shaft 20 via a distal joining member 41, a proximal end of the outer coil 30 is fixed to the shaft 20 via a proximal joining member 42, and a substantially middle portion (a medial portion) of the outer coil 30 that is located between the proximal joining member 42 and the distal joining member 41 is fixed to the shaft 20 via a middle joining member 43.

The materials used to form the distal joining member 41, the proximal joining member 42, and the middle joining member 43 are not particularly limited and include brazing metals such as Sn—Pb alloys, Pb—Ag alloys, Sn—Ag alloys, and Au—Sn alloys.

A first inner coil 52 and a second inner coil 54 are provided distal to the middle joining member 43 within the outer coil 30. The first inner coil 52 is provided within a distal portion of the outer coil 30, and the second inner coil 54 is provided proximal to the first inner coil 52.

In the guidewire 10, each of the first inner coil 52 and the second inner coil 54 is a single-strand coil formed by winding an elemental wire 52a or 54a, respectively, in a helical fashion. An outer diameter of the first inner coil 52 is substantially the same as an outer diameter of the second inner coil 54.

A distal end of the first inner coil 52 is fixed to the distal end of the shaft 20 via the distal joining member 41, while a proximal end of the first inner coil 52 is fixed to the shaft 20 via a first joining member 45. A distal end of the second inner coil 54 is fixed to the shaft 20 via a second joining member 46, while a proximal end of the second inner coil 54 is fixed to the shaft 20 via a third joining member 47. The first joining member 45 and the second joining member 46 are spaced from each other in the longitudinal direction N.

Usually, when torque is applied to a handling portion of the guidewire 10 and the outer coil 30 is tightened by winding, the outer coil 30 deforms inwardly, and the diameter of the outer coil 30 is reduced. When the outer coil 30 is excessively deformed, a winding of the elemental wire 32 may become displaced, sliding between adjacent windings of the elemental wire 32. However, the first inner coil 52 and the second inner coil 54 of the guidewire 10 are provided so as to support the outer coil and prevent excessive deformation. In particular, the first inner coil and the second inner coil are spaced from each other. Therefore, the first inner coil and the second inner coil do not interfere with each other when the outer coil deforms inwardly, contacting the inner coils.

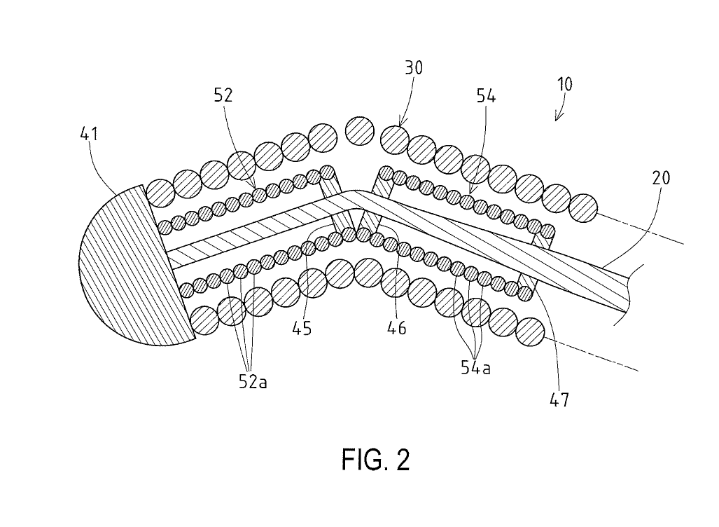

When the distal portion of the guidewire 10 presses against a hard calcified lesion, for example, it will bend. In the guidewire 10, as shown in FIG. 2, the first joining member 45 and the second joining member 46 come into contact with each other when the distal portion of the guidewire 10 is bent, preventing further movement of the first inner coil 52 and the second inner coil 54 and thus suppressing excessive bending. As a result, plastic deformation of the distal portion of the guidewire 10 is prevented. This ensures that the guidewire 10 can be reliably and consistently used even when the guidewire 10 is inserted into a hard calcified lesion.

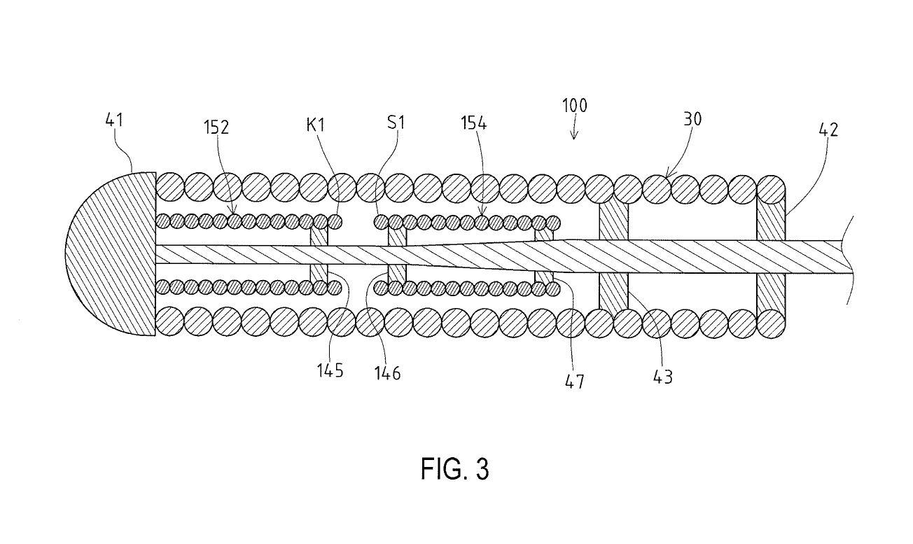

FIG. 3 is an expanded view of a partial cross-section of a guidewire 100 according to the disclosed embodiments. Throughout the drawings, like components are provided with the same reference numerals. The explanation below focuses on the differences between the guidewires shown in the drawings.

In the guidewire 10, the first joining member 45 joins the proximal end of the first inner coil 52, and the second joining member 46 joins the distal end of the second inner coil 54. On the other hand, in the guidewire 100, as shown in FIG. 3, a first joining member 145 joins a first inner coil 152 at a position within a proximal portion of the first inner coil 152 that is distal to a proximal end K1 of the first inner coil 152 (the most proximal winding of the first inner coil 152), and a second joining member 146 joins a second inner coil 154 at a position within a distal portion of the second inner coil that is proximal to a distal end S1 of the second inner coil 154 (the most distal winding of the second inner coil 154). In other words, the proximal end K1 of the first inner coil 152 projects proximally beyond the first joining member 145, and the distal end S1 of the second inner coil 154 projects distally beyond the second joining member 146.

When a distal portion of the guidewire 100 presses against a hard calcified lesion, for example, the first joining member 145 and the second joining member 146 come into contact with each when the distal portion of the guidewire is bent, preventing further movement of the first inner coil 152 and the second inner coil 154 and thus suppressing excessive bending. As a result, plastic deformation of the distal portion of the guidewire 100 is prevented.

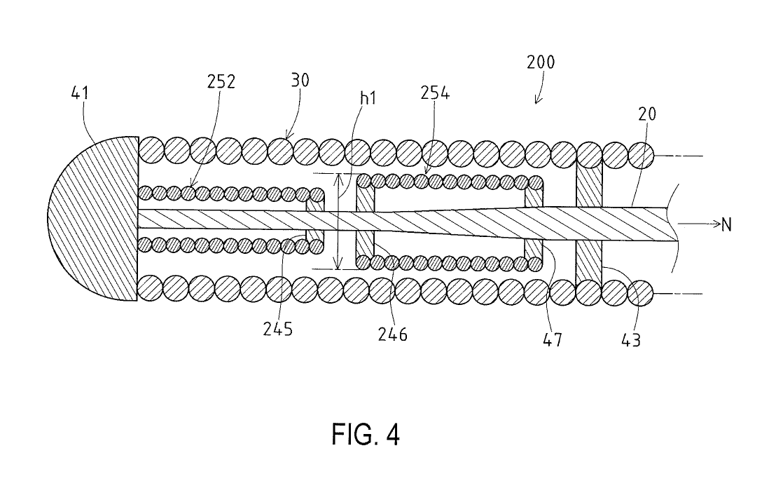

FIG. 4 is an expanded view of a partial cross-section of a guidewire 200 according to the disclosed embodiments. In the guidewire 10, an outer diameter of the first inner coil 52 is substantially the same as an outer diameter of the second inner coil 54. On the other hand, in the guidewire 200, an outer diameter of a second inner coil 254 is larger than an outer diameter of a first inner coil 252. In addition, a proximal end of the first inner coil 252 is fixed to the shaft 20 via a first joining member 245, and a distal end of the second inner coil 254 is fixed to the shaft 20 via a second joining member 246.

In the guidewire 200, the second joining member 246 has a width h1 (a height in the direction perpendicular to the longitudinal direction N) and a volume that are greater than the width and the volume of the first joining member 245. As a result, when a distal portion of the guidewire 200 presses against a hard calcified lesion and bends, a larger area of the second joining member 246 comes into contact with the first joining member 245. This further ensures that excessive bending of the guidewire 200 is suppressed, and plastic deformation of the distal portion of the guidewire 200 is effectively prevented.

In the guidewire 200, the outer diameter of the second inner coil 254 is larger than the outer diameter of the first inner coil 252, and the proximal end of the first inner coil 252 and the distal end of the second inner coil 254 are also spaced from each other in the direction perpendicular to the longitudinal direction N, and thus do not interfere with each other. Thus, the distal end of the second inner coil 254 can be efficiently joined to the shaft 20 after joining the proximal end of the first inner coil 252 without any interference occurring between the site of joining of the second inner coil 254 (the distal end of the second inner coil 254) and the proximal end of the first inner coil 252.

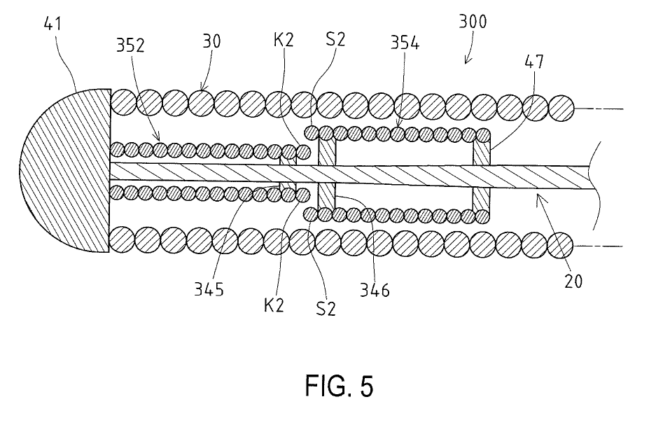

FIG. 5 is an expanded view of a partial cross-section of a guidewire 300 according to the disclosed embodiments. In the guidewire 200, the first joining member 245 joins the proximal end of the first inner coil 252 to the shaft 20, and the second joining member 246 joins the distal end of the second inner coil 254 to the shaft 20. On the other hand, in the guidewire 300, as shown in FIG. 5, a first joining member 345 joins a first inner coil 352 at a position within a proximal portion of the first inner coil 352 that is distal to a proximal end K2 of the first inner coil 352 (the most proximal winding of the first inner coil 352), and a second joining member 346 joins a second inner coil 354 at a position within a distal portion of the second inner coil 354 that is proximal to a distal end S2 of the second inner coil 354 (the most distal winding of the second inner coil 354). In other words, the proximal end K2 of the first inner coil 352 projects proximally beyond the first joining member 345, and the distal end S2 of the second inner coil 354 projects distally beyond the second joining member 346.

In the guidewire 300 having such a configuration, as in the case of the guidewire 100, when a distal portion of the guidewire 300 presses against a hard calcified lesion, the first joining member 345 and the second joining member 346 come into contact with each other, preventing further movement of the first inner coil 352 and second inner coil 354 and thus suppressing excessive bending of the distal portion of the guidewire 300. As a result, plastic deformation of the distal portion of the guidewire 300 is prevented.

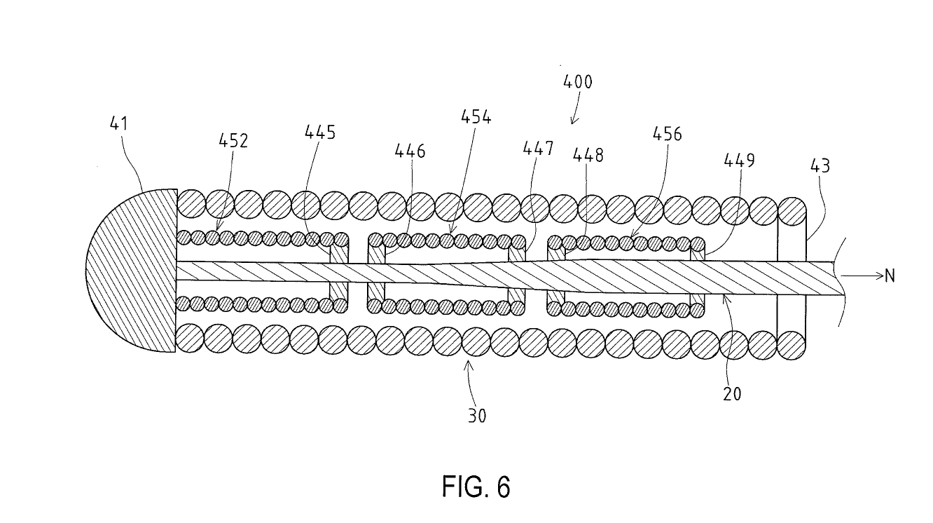

FIG. 6 is an expanded view of a partial cross-section of a guidewire 400 according to the disclosed embodiments. The guidewire 10 shown in FIG. 1 includes two inner coils: the first inner coil 52 and the second inner coil 54. On the other hand, the guidewire 400 includes three inner coils: a first inner coil 452, a second inner coil 454, and a third inner coil 456, in this order from the distal end of the guidewire 400.

A proximal end of the first inner coil 452 is joined to the shaft 20 via a first joining member 445. A distal end of the second inner coil 454 is joined to the shaft 20 via a second joining member 446, and a proximal end of the second inner coil 454 is joined to the shaft 20 via a third joining member 447. A distal end of the third inner coil 456 is joined to the shaft 20 via a fourth joining member 448, and a proximal end of the third inner coil 456 is joined to the shaft 20 via a fifth joining member 449. The first joining member 445 and the second joining member 446 are spaced from each other in the longitudinal direction N, and the third joining member 447 and the fourth joining member 448 are spaced from each other in the longitudinal direction N.

When the distal portion of the guidewire 400 presses against a hard calcified lesion and bends, not only the interaction between the first joining member 445 and the second joining member 446 but also the interaction between the third joining member 447 and the fourth joining member 448 suppresses excessive bending. As a result, plastic deformation of the distal portion of the guidewire is prevented more effectively.

Although the guidewire 400 includes three inner coils, the number of inner coils is not limited to three and may be four or greater.

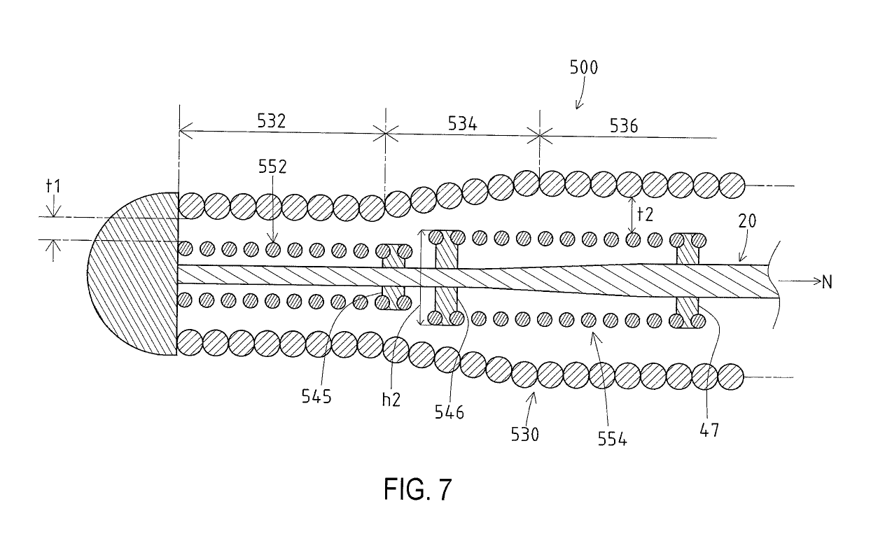

FIG. 7 is an expanded view of a partial cross-section of a guidewire 500 according to the disclosed embodiments. In the guidewire 10, the outer coil 30 has an outer diameter that is uniform in the longitudinal direction. On the other hand, in the guidewire 500, an outer coil 530 has an outer diameter that increases from a distal end of the outer coil 530 toward a proximal end of the outer coil 530. More particularly, the outer coil 530 includes a first linear portion 532, a tapered portion 534, and a second linear portion 536 that has an outer diameter larger than an outer diameter of the first linear portion 532, in this order from the distal end of the outer coil 530 toward the proximal end of the outer coil 530.

This makes it possible, for example, to easily insert a distal portion (the first linear portion 532) of the guidewire 500 into a narrowed lesion and then to smoothly proceed deeper into the tubular organ or body tissue by gradually pushing and expanding the narrowed lesion with the tapered portion 534.

In the guidewire 500, a second inner coil 554 has an outer diameter that is larger than an outer diameter of a first inner coil 552. A clearance t1 between the first inner coil 552 and the outer coil 530 (the first linear portion 532) is substantially the same as a clearance t2 between the second inner coil 554 and the outer coil 530 (the second linear portion 536). The clearance t1 (t2) is 5 to 10 μm, which is enough to suppress inward deformation of the outer coil 530 when the outer coil 530 is twisted in a manner that tightens the outer coil 530.

In addition, the outer diameter of the first inner coil 552 and the outer diameter of the second inner coil 554 correspond to the shape of the outer coil 530 having a diameter that increases from the distal end of the outer coil 530 toward the proximal end of the outer coil 530, and are spaced from the outer coil 530 by the clearance t1 (t2) that is enough to suppress inward deformation of the outer coil 530. Additionally, because of the difference in outer diameters, a proximal end of the first inner coil 552 and a distal end of the second inner coil 554 are spaced from each other in the direction perpendicular to the longitudinal direction N, and thus do not interfere with each other.

Thus, the distal end of the second inner coil 554 can be efficiently joined to the shaft 20 after joining the proximal end of the first inner coil 552 without any interference occurring between the site of joining of the second inner coil 554 (the distal end of the second inner coil 554) and the proximal end of the first inner coil 552.

In addition, in the guidewire 500, the second joining member 546 has a width h2 (a height in the direction perpendicular to the longitudinal direction N) and a volume that are greater than the width and the volume of the first joining member 545. As a result, when a distal portion of the guidewire 500 presses against a hard calcified lesion and bends, a larger area of the second joining member 546 comes into contact with the first joining member 545. This further ensures that excessive bending of the guidewire 500 is suppressed, and plastic deformation of the distal portion of the guidewire 500 is effectively prevented.

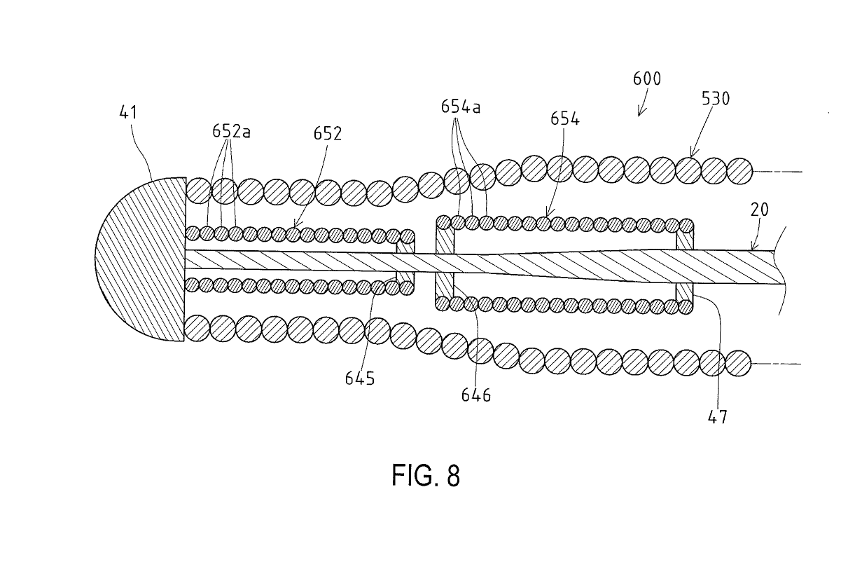

FIG. 8 is an expanded view of a partial cross-section of a guidewire 600 according to the disclosed embodiments. In the guidewire 500, the first inner coil and the second inner coil are each a single-strand coil formed by winding an elemental wire in a helical fashion. On the other hand, in the guidewire 600, a first inner coil 652 and a second inner coil 654 are each formed by winding a plurality of elemental wires.

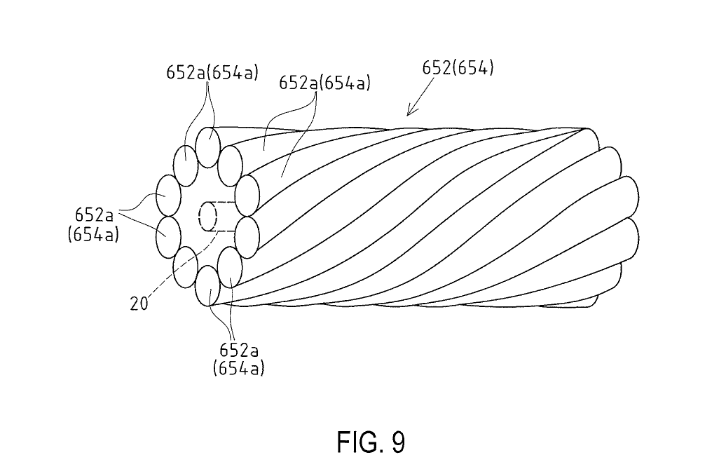

As shown in FIG. 9, the first inner coil 652 and the second inner coil 654 are each a hollow inner coil formed by winding a plurality of elemental wires 652a (654a) (ten elemental wires 652a (654a) in the guidewire 600). This allows a joining material used to join the inner coils 652, 654 to the shaft 20 to preferentially penetrate into gaps between the elemental wires 652a (654a) along the longitudinal direction by capillary action. As a result, the resulting joining member (the first joining member 645 and the second joining member 646) has an adequate volume and rigidity.

Therefore, when the distal portion of the guidewire 600 presses against a hard calcified lesion and bends, the first joining member 645 and the second joining member 646 come into contact with each other and prevent excessive bending. As a result, plastic deformation of the distal portion of the guidewire 600 is prevented more reliably.

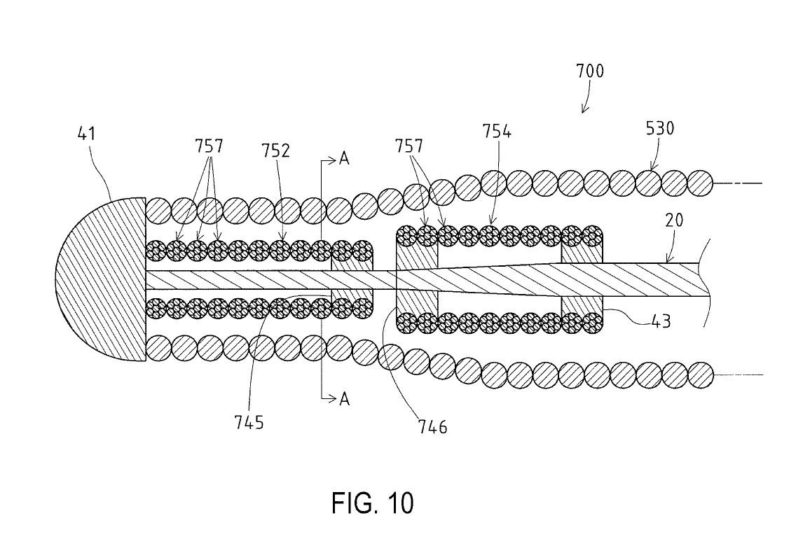

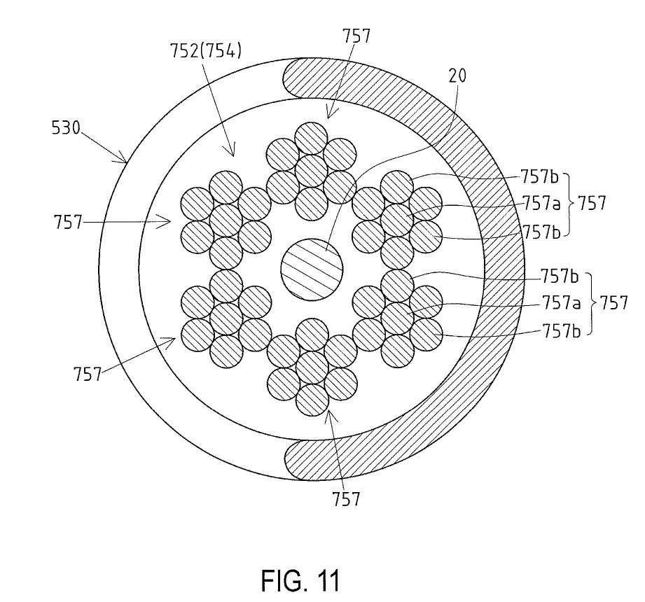

FIG. 10 is an expanded view of a partial cross-section of a guidewire 700 according to the disclosed embodiments. As shown in FIGS. 10 and 11, a first inner coil 752 and a second inner coil 754 of the guidewire 700 are each formed by winding a plurality of stranded wires 757 in a helical fashion, each of the stranded wires 757 being formed by twisting a plurality of elemental wires together. More specifically, as shown in FIG. 11, each of the first inner coil 752 and the second inner coil 754 is formed by winding six stranded wires 757 in a helical fashion, each of the stranded wires 757 being formed by twisting a core wire 757a and six peripheral wires 757b wound around the outer circumference of the core wire 757a.

The materials used to form the core wire 757a and the peripheral wires 757b in the first inner coil 752 and the second inner coil 754 are not particularly limited and include stainless steels such as martensitic stainless steel, ferritic stainless steel, austenitic stainless steel, austenitic-ferritic duplex stainless steel, and precipitation hardened stainless steel; super-elastic alloys such as Ni—Ti alloys; and metals radiopaque to X-rays, such as platinum, gold, tungsten, tantalum, iridium, and alloys thereof

Because the first inner coil 752 and the second inner coil 754 are each formed by winding a plurality of stranded wires 757 in a helical fashion, and each of the stranded wires 757 is formed by twisting a plurality of elemental wires 757a and 757b together, the stranded wires 757 are in close contact with each other and therefore gaps between the elemental wires 757a, 757b are small. This further facilitates penetration of the joining material into the gaps between the elemental wires along the longitudinal direction N when forming joining members 745 and 746. As a result, the joining members 745 and 746 have large volumes and enhanced rigidity compared to the joining members 645 and 646 of the guidewire 600.

Therefore, when the distal portion of the guidewire 700 presses against a hard calcified lesion and bends, the first joining member 745 and the second joining member 746 come into contact with each other and even more surely prevent excessive bending. As a result, plastic deformation of the distal portion of the guidewire 700 is prevented even more reliably.

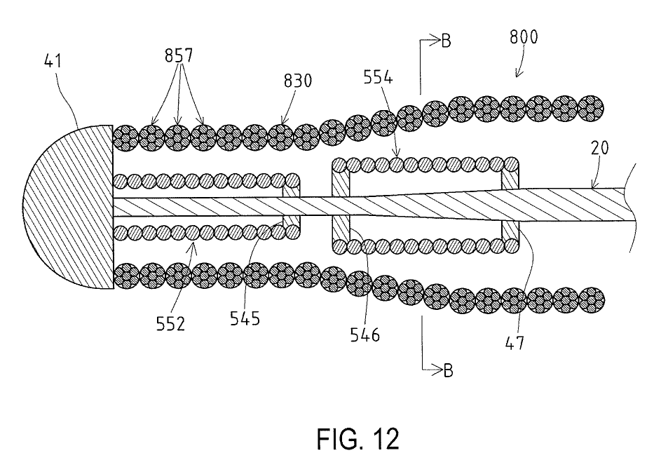

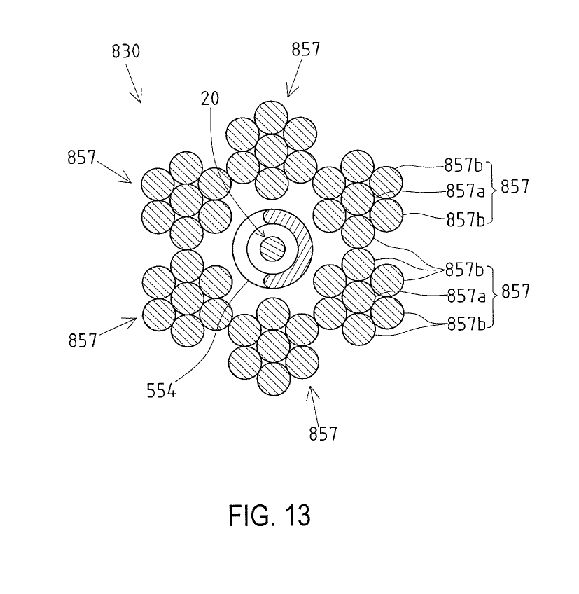

FIG. 12 is an expanded view of a partial cross-section of a guidewire 800 according to the disclosed embodiments. In the guidewire 500, the outer coil 530 is a single-strand coil formed by winding an elemental wire in a helical fashion. On the other hand, as shown in FIG. 12, an outer coil 830 of a guidewire 800 is formed by winding a plurality of stranded wires 857 in a helical fashion, each of the stranded wires 857 being formed by twisting a plurality of elemental wires together. More specifically, as shown in FIG. 13, the outer coil 830 is formed by winding six stranded wires 857 in a helical fashion, each of the stranded wires 857 being formed by twisting a core wire 857a and six peripheral wires 857b wound around the outer circumference of the core wire 857a.

The materials used to form the core wire 857a and the peripheral wires 857b in the outer coil 830 are not particularly limited and include stainless steels such as martensitic stainless steel, ferritic stainless steel, austenitic stainless steel, austenitic-ferritic duplex stainless steel, and precipitation hardened stainless steel; super-elastic alloys such as Ni—Ti alloys; and metals radiopaque to X-rays, such as platinum, gold, tungsten, tantalum, iridium, and alloys thereof.

As discussed above, the outer coil 830 is formed by winding a plurality of stranded wires 857 in a helical fashion, each of the stranded wires 857 being formed by twisting a plurality of elemental wires together. The elemental wires 857a and 857b are capable of slightly moving relative to each other, providing the outer coil 830 with excellent flexibility and an adequate restoring capacity. As a result, when a distal portion of the guidewire 800 presses against a hard calcified lesion and bends, the shape of the guidewire 800 can be manually restored with ease, which makes it possible to use the guidewire 800 consistently.

Furthermore, when torque is applied to a handling portion of the guidewire 800, not only are the elemental wires 857a, 857b pressed against each other, but also the stranded wires 857 are pressed against each other, increasing contact pressure and enhancing close contact. This improves torque transmission and ensures excellent maneuverability of the guidewire 800.

Although the features of the guidewire 800 have been described in comparison to the guidewire 500 as an example, the outer coil 830 may be used in the other described guidewires 10, 100, 200, 300, 400, 600, 700. The outer coil 830 does not adversely affect the functionality of the guidewires 10, 100, 200, 300, 400, 500, 600, 700, 800. Instead, the outer coil 830 makes it possible to ensure consistent use of the guidewire 10, 100, 200, 300, 400, 500, 600, 700, 800, improve torque transmission, and ensure excellent maneuverability.

What is claimed is:

1. A guidewire comprising:
a shaft;
an outer coil provided around an outer circumference of a distal portion of the shaft;
a first inner coil provided within a distal portion of the outer coil; and
a second inner coil provided proximal to the first inner coil within the outer coil,
wherein:
a proximal end of the first inner coil is joined to the shaft via a first joining member, and a distal end of the second inner coil is joined to the shaft via a second joining member, and
the first joining member and the second joining member are spaced from each other in a longitudinal direction of the guidewire.

2. The guidewire according to claim 1, wherein the outer coil has an outer diameter that increases from a distal end of the outer coil toward a proximal end of the outer coil.

3. The guidewire according to claim 1, wherein a diameter of the second inner coil is greater than a diameter of the first inner coil.

4. The guidewire according to claim 2, wherein a diameter of the second inner coil is greater than a diameter of the first inner coil.

5. The guidewire according to claim 1, wherein each of the first inner coil and the second inner coil is formed by winding a plurality of elemental wires together.

6. The guidewire according to claim 2, wherein each of the first inner coil and the second inner coil is formed by winding a plurality of elemental wires together.

7. The guidewire according to claim 3, wherein each of the first inner coil and the second inner coil is formed by winding a plurality of elemental wires together.

8. The guidewire according to claim 1, wherein the outer coil is formed by winding a plurality of elemental wires together.

9. The guidewire according to claim 1, wherein the outer coil is formed by winding a plurality of stranded wires in a helical fashion, each of the stranded wires being formed by twisting a plurality of elemental wires together.

10. The guidewire according to claim 2, wherein the outer coil is formed by winding a plurality of stranded wires in a helical fashion, each of the stranded wires being formed by twisting a plurality of elemental wires together.

11. The guidewire according to claim 3, wherein the outer coil is formed by winding a plurality of stranded wires in a helical fashion, each of the stranded wires being formed by twisting a plurality of elemental wires together.

12. The guidewire according to claim 4, wherein the outer coil is formed by winding a plurality of stranded wires in a helical fashion, each of the stranded wires being formed by twisting a plurality of elemental wires together.

13. The guidewire according to claim 5, wherein the outer coil is formed by winding a plurality of stranded wires in a helical fashion, each of the stranded wires being formed by twisting a plurality of elemental wires together.

14. The guidewire according to claim 6, wherein the outer coil is formed by winding a plurality of stranded wires in a helical fashion, each of the stranded wires being formed by twisting a plurality of elemental wires together.

15. The guidewire according to claim 7, wherein the outer coil is formed by winding a plurality of stranded wires in a helical fashion, each of the stranded wires being formed by twisting a plurality of elemental wires together.

16. The guidewire according to claim 1, wherein each of the first inner coil and the second inner coil is formed by winding a plurality of stranded wires in a helical fashion, each of the stranded wires being formed by twisting a plurality of elemental wires together.

17. The guidewire according to claim 1, wherein a distal end of the first inner coil is joined to a distal end of the outer coil via a distal joining member.

18. The guidewire according to claim 1, further comprising:
a third inner coil provided proximal to the second inner coil within the outer coil,
wherein:
a proximal end of the second inner coil is joined to the shaft via a third joining member, a distal end of the third inner coil is joined to the shaft via a fourth joining member, and a proximal end of the third inner coil is joined to the shaft via a fifth joining member, and
the third joining member and the fourth joining member are spaced from each other in the longitudinal direction of the guidewire.

19. A guidewire comprising:
a shaft;
an outer coil provided around an outer circumference of a distal portion of the shaft;
a first inner coil provided within a distal portion of the outer coil; and
a second inner coil provided proximal to the first inner coil within the outer coil;
wherein:
a proximal portion of the first inner coil is joined to the shaft via a first joining member, and a proximal end of the first inner coil projects proximally beyond the first joining member,
a distal portion of the second inner coil is joined to the shaft via a second joining member, and a distal end of the second inner coil projects distally beyond the second joining member, and
the proximal end of the first inner coil and the distal end of the second inner coil are spaced from each other in a longitudinal direction of the guidewire.

20. The guidewire according to claim 19, wherein:
the first joining member joins the proximal portion of the first inner coil to the shaft at a winding of the first inner coil adjacent to a most proximal winding of the first inner coil, and
the second joining member joins the distal portion of the second inner coil to the shaft at a winding of the second inner coil adjacent to a most distal winding of the second inner coil.
